(19) United States
(12) Patent Application Publication
(10) Pub. No.: US 2009/0221665 A1
(43) Pub. Date: Sep. 3, 2009

(54) Organic Compounds

(76) Inventor: David Lewis Earnest, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/086,656

(22) PCT Filed: Dec. 20, 2006
(86) PCT No.: PCT/EP2006/012312

§ 371 Date: Jun. 17, 2008

(30) Foreign Application Priority Data
Dec. 22, 2005 (GB) 0526258.9

(51) Int. Cl. A61K 31/405; A61K 31/22

(52) U.S. Cl. 514/415; 514/551

(57) ABSTRACT

The present invention relates to a combination of a 5-HT4 receptor agonist and a cholinesterase inhibitor and pharmaceutical compositions and formulations containing the combination. The pharmaceutical combination may be employed for the treatment of altered gastrointestinal motility, sensitivity, secretion or abdominal disorders. The dosage is preferably oral. The preferred 5-HT₄ receptor agonist is tegaserod.

The present invention relates to pharmaceutical combinations comprising or 5-HT₄ receptor agonist and a cholinesterase inhibitor, and their uses in treating gastrointestinal and other disorders.

Serotonin (5-hydroxytryptamine; 5-HT) functions as a neurotransmitter in the mammalian central nervous system (CNS) and in the periphery. Serotonin is one of the transmitters to be recognized for its physiological importance, and agents which interact with 5-HT receptors have been the focus of much research (P. Bonate, Clinical Neuropharmacology, 1991, Vol. 14(1), pp. 1-16). To-date, the number of serotonin receptor subtypes identified is into the tens, including the major classes, e.g., 5-HT₁, 5-HT₂, 5-HT₃, 5-HT₄, 5-HT₅, 5-HT₆, and 5-HT₇. Because of the multiplicity of serotonin receptor subtypes, the identification of which serotonin receptor subtype is correlated to various physiological/pharmacological actions is complicated.

A number of gastrointestinal syndromes are related to the production and actions of serotonin, and they have a fairly common occurrence in a very large number of people worldwide. Some of the more well-known gastrointestinal conditions, syndromes or diseases are IBS, gastro-esophageal reflux disease (“GERD”) and dyspepsia.

IBS is a chronic condition associated with abdominal pain, bloating and altered bowel function and is estimated to affect as much as 10-20% of the population. Sometimes the disease is referred to as irritable colon, spastic colon, spastic colitis or mucous colitis. The latter two are almost certainly misnomers, as colitis implies inflammation of the colon, and an absence of inflammation is one of the defining observations in a diagnosis of IBS. The cause of IBS is unknown, but a number of factors have been implicated, including diet, lifestyle, depression, anxiety, infections and unrelated inflammatory conditions, including early life insult resulting in central neuronal sensitization and sensitizing of neurons in the gut.

IBS may be associated with, for example, diarrhea, mixed or alternating bowel habits, or constipation. Prolonged constipation is known as chronic constipation; symptoms may also include abdominal discomfort, bloating, and straining.

GERD is a condition that is associated with the reflux of gastric contents to the esophagus through the lower esophageal sphincter. GERD is characterized by symptoms of heartburn, bloating, abdominal pain, epigastric pain, early satiety, nausea, regurgitation, burbulence and vomiting. The reflux is thought to occur because of an increased incidence of transient lower esophageal sphincter relaxations allowing gastric contents to enter the esophagus.

Dyspepsia is also an important health problem. The most common conditions that are associated with patients who present with chronic symptoms of dyspepsia are GERD, duodenal ulcer or gastric ulcer and other diagnoses (e.g. functional/non-ulcerative dyspepsia, gallbladder or liver disease).

These conditions or diseases are characterized by altered motility, sensitivity, secretion and/or infections with Helicobacter pylori as well as potentially a psychological (usually subconscious) overlay. At present, only few medications have shown clinically significant efficacy for the treatment of, e.g., functional dyspepsia.

Prokinetic agents act to facilitate neurotransmission in the cholinergic system of the bowel. Acetylcholine (ACh) is the primary excitatory neurotransmitter on the enteric ganglia producing stimulation of contractile activity and secretion. ACh is released from pre-synaptic nerve endings and travels across the synapse to the post-synaptic cell where it binds to and activates specific receptors. It is subsequently enzymatically degraded by the enzyme acetylcholinesterase. An acetylcholinesterase inhibitor, also known as a cholinesterase inhibitor, suppresses the action of the enzyme and can achieve an enhances level of ACh in the cholinergic synapse.

It has now been found that a combination comprising a 5-HT₄ receptor agonist, e.g., as defined below, and a cholinesterase inhibitor, e.g., as defined below, has a beneficial effect and is useful in the treatment of altered gastrointestinal motility, sensitivity and/or secretion and/or abdominal disorders. This combination may also be used to regulate, stabilize and normalize altered gastrointestinal motility, sensitivity and/or secretion and/or abdominal disorders.

### DEFINITIONS

Unless otherwise specified herein, common definitions are intended by the words and terms used herein. For example, the term “pharmaceutical combination” as used herein means a product that results from the mixing or combining of more than one active ingredient and includes both fixed and non-fixed combinations of the active ingredients.

The term “fixed combination” as that term is used herein means that the active ingredients, e.g. tegaserod and a cholinesterase inhibitor, are both administered to a patient simultaneously in the form of a single entity or dosage. As an example, a fixed combination would be one capsule containing two active ingredients.

The term “non-fixed combination” as that term is used herein means that the active ingredients, e.g. tegaserod and a cholinesterase inhibitor, are both administered to a patient as separate entities either simultaneously, concurrently or sequentially with no specific time limits, wherein such administration provides therapeutically effective levels of the two compounds in the body, preferably at the same time. As an example, a non-fixed combination would be two capsules each containing one active ingredient where the purpose is to have the patient achieve treatment with both active ingredients together in the body.

The term “altered gastrointestinal motility, sensitivity and/or secretion disorder(s)” as used herein includes one or more of the symptoms and conditions which affect the gastrointestinal tract from the mouth to the anus, which include, but are not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, gastroparesis, e.g. diabetic gastroparesis, ulcerative colitis, Crohn's disease, ulcers and the visceral pain associated therewith.

The term “abdominal disorder(s)” as used herein includes those conditions which affect the lower abdomen and include but are not limited to those conditions treated by regulation, stabilization and normalization of enterochromaffin cell functions, GI secretion, motility, afferent and efferent fiber activity and/or abdominal smooth muscle cell activity.

The term “gastro-esophageal reflux disease” and “GERD” as used herein means the incidence of, and the symptoms of, those conditions caused by the reflux of the stomach contents into the esophagus. This includes all forms/manifestations of GERD including, but not limited to, erosive and non-erosive GERD, heartburn and other symptoms associated with GERD.

The term “irritable bowel syndrome” and “IBS” as used herein means a disorder of function involving altered motility, sensitivity and secretion involving primarily the small intestine and large bowel associated with variable degrees of abdominal pain, bloating, constipation or diarrhea without overt bowel inflammation.

The term “dyspepsia” as used herein means a condition characterized by symptoms of epigastric pain, abdominal pain, bloating, early satiety, nausea, heartburn and vomiting as a primary gastrointestinal dysfunction or as a complication due, and not exclusive to disorders such as ulcer disease, appendicitis, gallbladder disturbances, or malnutrition.

The term “gastroparesis” as used herein means a paralysis of the stomach brought about by a motor abnormality in the stomach which is often manifested as delayed gastric emptying. This can also be a complication of diseases such as diabetes, progressive systemic sclerosis, anorexia nervosa, or myotonic dystrophy.

The term “constipation” as used herein means a condition characterized by infrequent and/or difficult evacuation of feces resulting from conditions such as altered GI motility, altered sensation or evacuation functions, altered secretion or reabsorption of electrolytes and water.

The term “diarrhea” as used herein means a condition characterized by frequent evacuations of feces often associated with large volumes and urge resulting from conditions such as altered GI motility, altered sensation and secretion or reabsorption of electrolytes and water.

The term “treat” or “treatment” encompasses the complete range of therapeutically positive effects associated with pharmaceutical medication including reduction of, alleviation of and relief from the symptoms or illness which affect the organism.

### DESCRIPTION

The present invention provides a pharmaceutical combination comprising:
a) a 5-HT4 receptor agonist, or a pharmaceutically acceptable salt, racemate or enantiomer thereof; and
b) a cholinesterase inhibitor, or a pharmaceutically acceptable salt, racemate or enantiomer thereof.

5-HT₄ receptor agonists are agents that have an affinity for serotonin type-4 receptors, and are able to mimic the stimulating effects of serotonin at the cellular receptor; they include any compound which can partially activate 5-HT₄ receptors (intrinsic activity less than that of serotonin, i.e. <1.00. The intrinsic activity may be determined in the non-electrically or electrically stimulated guinea pig ileum or striatum assay, e.g. as disclosed in EP-A1-0 505 322, Br. J. Pharmacol., 115, 1387, 1995 or in the guinea pig distal colon test e.g. as disclosed in Br. J. Pharm., 1593-1599, 1993).

Examples of 5-HT₄ receptor agonists include tegaserod, zacopride, prucalopride, mosapride, nircisapride, E3620, ABT224, VI0134, AT17505, and TD2749.

In one embodiment, the 5-HT₄ receptor agonist is selected from a compound of formula I:

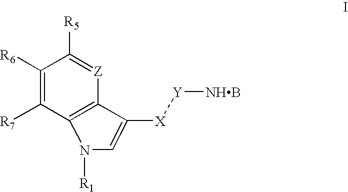

wherein
R₁ is hydrogen; C₁₋₆alkyl; (C₁₋₆alkyl)carbonyl; benzoyl; or phenylC₁₋₄alkyl-carbonyl;
R₅ is hydrogen; halogen; C₁₋₆alkyl; hydroxy; nitro; amino; C₁₋₆alkylamino; C₁₋₁₀alkyl-carbonylamino; C₂₋₆alkoxycarbonyl; SO₂NRₐR_{b} wherein each of Rₐ and R_{b} independently is hydrogen or C₁₋₆alkyl; cyano; or trimethylsilyl; C₁₋₆alkyl substituted by —SO₂—C₁₋₆alkyl, —SO₂NRₐR_{b}, —CONRₐR_{b}, —NH—SO₂—C₁₋₆alkyl, —N(C₁₋₆alkyl)-SO₂—(C₁₋₆alkyl), —NRₐR′_{b} wherein R′_{b} is hydrogen or C₁₋₆alkyl, C₂₋₆alkoxycarbonyl or —PO(C₁₋₄alkyl)₂; carboxy; CONRₐR_{b}; —PO(C₁₋₆alkyl)₂; OCONR_{c}R_{d}, wherein each of R_{c} and R_{d} independently is C₁₋₆alkyl;
R₆ is hydrogen or, when R₅ is OH, R₆ is hydrogen or halogen,
Z is —CR₄═ wherein R₄ is hydrogen, halogen, hydroxy or C₁₋₆alkyl or, when R₅ is hydrogen or hydroxy, Z is also —N═,
R₇ is hydrogen, halogen, C₁₋₆alkyl or C₁₋₆alkoxy,
X-Y is —CR₈═N— or —CH(R₈)—NH— wherein R₈ is hydrogen or C₁₋₆alkyl, and
B is a radical of formula (a) or (b),

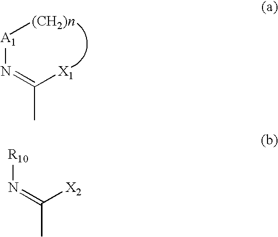

wherein
n is 1 or 2,
A₁ is C═O or CH₂,
X₁ is S; NR₁₁ wherein R₁₁ is hydrogen, (C₁₋₆alkyl)carbonyl, benzoyl or phenylC₁₋₄alkyl-carbonyl; or CR₁₂R₁₃ wherein each of R₁₂ and R₁₃ independently is hydrogen or C₁₋₄alkyl,
R₁₀ is hydrogen; C₁₋₁₂alkyl; C₁₋₆alkyl substituted by hydroxy, aryl, aryloxy, adamantyl, a heterocyclic radical, —NR₁₅—CO—R₁₆ or —NH—SO₂-aryl; C₅₋₇cycloalkyl; adamantyl; (C₁₋₁₀alkyl)carbonyl; benzoyl; phenyl(C₁₋₄alkyl)carbonyl; or —CONHR₁₄,
   wherein
R₁₄ is C₁₋₁₀alkyl or C₅₋₇cycloalkyl,
R₁₅ is hydrogen or C₁₋₄alkyl, and
R₁₆ is C₁₋₆alkyl, C₅₋₇cycloalkyl, C₅₋₇cycloalkyl-C₁₋₄alkyl, aryl or arylC₁₋₄alkyl,
wherever “aryl” appears as is or in the significances “aryloxy”, “—NH—SO₂-aryl” or “aryl(C₁₋₄alkyl)” in the above definition, it is phenyl or phenyl substituted by halogen, C₁₋₄alkyl or C₁₋₆alkoxy; and
wherever “heterocyclic radical” appears in the above definition, it is pyridyl, imidazolyl, benzimidazolyl, pyrrolidinyl, pyrrolidonyl, piperidino, pyrazinyl, perhydroindolyl or a radical of formula (c), (d) or (e)

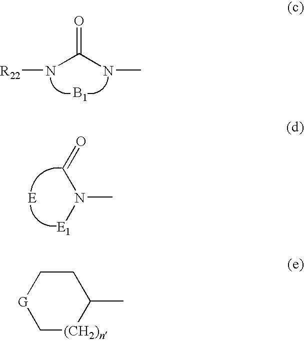

wherein
R₂₂ is hydrogen or C₁₋₄alkyl,
B₁ is —CH₂CH₂—, —COCH₂— or —(CH₂)₃— in which one or two H thereof can by replaced by C₁₋₄alkyl, or 1,2-phenylene,
E is —CH₂—CH₂—, —CH₂N(R₁₇)— or —(CH₂)₃— in which one or two H thereof can be replaced by C₁₋₆alkyl, or 1,2-phenylene,
E₁ is CO or CH₂,
R₁₇ is hydrogen or C₁₋₄alkyl,
G is CO, —CHCOOR₁₈, —CHCOR₁₉, 5,5-dimethyl-1,3-dioxan-2-ylidene or 1,3-dioxolan-2-ylidene, wherein R₁₈ is hydrogen or C₁₋₆alkyl and R₁₉ is C₁₋₄alkyl, and
n′ is 0 or 1, and
X₂ is —SR₂₀ or —NR₃R′₁₀ wherein R₂₀ is C₁₋₆-alkyl, R₃ is hydrogen or C₁₋₆-alkyl and R′₁₀ has one of the significances given for R₁₀ above, or R₃ and R′₁₀ together with the nitrogen atom to which they are attached form a heterocyclic radical as defined above;
with the proviso that where B is a radical of formula (b), only one of R₁₀ and R′₁₀ can be other than hydrogen and X₂ can be —SR₂₀ only when R₁₀ is hydrogen,
   and a physiologically-hydrolysable and -acceptable ether or ester thereof when R₅ is hydroxy,
   in free form or in salt form.

Compounds of formula I and their physiologically-hydrolysable and -acceptable ethers or esters are e.g. as disclosed in EP-A1-0 505 322. Suitable pharmaceutically acceptable salts are, e.g., pharmaceutically acceptable acid addition salts, for example such salts as obtained with an inorganic or organic acid, e.g. the hydrochloride, sulfate, acetate, oxalate, maleate and fumarate salts.

By the term “physiologically-hydrolysable and -acceptable ethers or esters” as applied to the compounds of formula I when R₅ is hydroxy, is meant ethers in which R₅ is etherified (e.g. by optionally substituted C₁₋₆alkyl) and esters in which R₅ is esterified and which are hydrolysable under physiological conditions to yield an alcohol or acid which is physiologically acceptable, i.e. which is non-toxic at the desired dosage levels. Specific examples are given in EP-A1-0 505 322.

Preferred compounds of formula I as 5-HT₄ receptor partial agonists are e.g. those wherein R₁ is H, Z is —CH═ and R₅ is OH or C₁₋₆alkoxy.

Further examples of 5-HT₄ receptor partial agonists include e.g. RS 67333 (1-(4-amino-5-chloro-2-methoxyphenyl)-3-[1-butyl-4-piperidinyl]-1-propanone), or RS 67506 (1-(4-amino-5-chloro-2-methoxyphenyl)-3-[1-methylsulphonylamino)ethyl-4-piperidinyl]-1-propanone).

A particularly preferred compound of formula I is the compound of formula

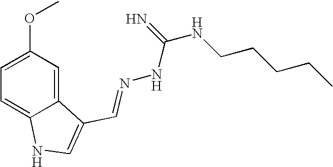

in free form or in pharmaceutically acceptable salt form. This compound has the chemical name of 3-(5-methoxy-1H-indol-3-yl-methylene)-N-pentylcarbazimidamide, and is also known as tegaserod. It is disclosed as being a 5-HT₄ receptor partial agonist. It may also exist in form of tautomers

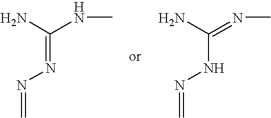

which are included in the present invention. A preferred salt form is the hydrogen maleate. A preferred crystalline form is as described in WO 2005/014544.

Suitable acetylcholinesterase inhibitors are compounds that can inhibit the activity of the enzyme acetylcholinesterase, as determined using the methods of Ellmann (Ellmann, G L et al, 1961, A new and rapid calorimetric determination of acetylcholinesterase activity, biochemical Pharmacology, 7:88-95), or modifications of these (e.g. Barr et al, Americal Journal of Hematology, 28(4):260-5, 1988 August). Several compounds of this class are in clinical use (reviewed by Krall et al., 1999, Annals of Pharmacology 33:441-450; Jann et al Clinical Pharmacokinetics 41(10):719-739), primarily for the treatment of Alzheimer's Disease and Myaesthenia Gravis, where cholinergic transmission is compromised.

Examples of acetylcholinesterase inhibitors include agents such as neostigmine, physostigmine, tacrine, donepezil, rivastigmine, metrifonate, galamantine, and pyridostigmine.

Preferred cholinesterase inhibitors are those set forth in U.S. Pat. No. 4,948,807, more preferably rivastigmine tartrate (Exelon®); U.S. Pat. No. 4,895,841, more preferably donepezil hydrochloride (Aricept®); and U.S. Pat. No. 4,663,318, more preferably galanthamine hydrobromide (Reminyl®). Pyridostigmine Bromide (Mestinon®) is another preferred cholinesterase inhibitor.

Most preferred is rivastigmine, particularly as rivastigmine tartrate (Exelon®).

A preferred combination of the invention is one which comprises tegaserod, e.g. in hydrogen maleate salt form, formulated as a solid oral pharmaceutical composition, e.g. a tablet. Preferred solid oral pharmaceutical compositions are described in WO 00/10526 and WO 03/053432.

In accordance with another aspect of the invention there is provided a pharmaceutical composition comprising a combination of a 5-HT4 receptor agonist and a cholinesterase inhibitor as active ingredients, or pharmaceutically acceptable salts, racemates or enantiomers thereof, in the presence of a pharmaceutically acceptable carrier, and optionally, other therapeutic ingredients. In a preferred embodiment the 5-HT₄ receptor I agonist is a compound of formula I as defined above having an intrinsic activity <1.00, e.g. a compound of formula I wherein R₁ is H, Z is —CH═ and R₅ is OH or C₁₋₆alkoxy; in an even more preferred embodiment the first agent is tegaserod, preferably in the hydrogen maleate salt form.

The terms “pharmaceutically acceptable salts” or “a pharmaceutically acceptable salt thereof” refer to salts prepared from pharmaceutically acceptable nontoxic acids or bases including inorganic acids and bases. Suitable pharmaceutically acceptable acid addition salts for the first agent and the cholinesterase inhibitors of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like.

To prepare the pharmaceutical compositions of the present invention, the 5-HT₄ receptor agonist and a cholinesterase inhibitor, or their pharmaceutically acceptable salts, racemates or enantiomers are combined in intimate admixture by mixing, blending or combining in any manner known to those of skill in the art, with a pharmaceutically acceptable carrier. The pharmaceutically acceptable carrier may take a wide variety of forms depending on the form of preparation desired for administration.

Any suitable route of administration may be employed for providing a mammal with a therapeutically effective amount of the pharmaceutical combinations and compositions of the present invention. For example, oral, rectal, vaginal, topical, parental (subcutaneous, intramuscular, intravenous, transdermal) and like forms of administration may be employed. Dosage formulations include ointments, foams, gels, transdermal patches, tablets (both fractionable and non-fractionable), caplets, powders for inhalations, gelcaps, capsules, elixirs, syrups, chewable tablets, lozenges, troches, dispersions, aerosols, solutions, fast-dissolving wafers, suppositories or suspensions or other known and effective delivery methods.

In addition to the dosage formulations set out above, the pharmaceutical combinations and compositions of the present invention may also be administered by controlled release means and/or delivery devices such as those described in U.S. Pat. Nos. 3,845,770; 3,916,899; 3,536,809; 3,598,123; and 4,008,719 and by “fast-melt” means which include delivery devices which rapidly dissolve in the mouth. Rapid dissolution is meant to include dissolution which takes place in the patient's mouth within less than three minutes. Delivery devices for this type of formulation include, but are not limited to, tablets and capsules. An example of a fast-melt means as used herein is described in U.S. Pat. No. 5,178,878 which discloses an effervescent dosage form with microparticles for rapid dissolution of the tablet or capsule.

Oral dosing is preferred. In preparing the compositions in oral dose form, any of the usual pharmaceutical carriers may be employed including any material, composition or vehicle, such as a liquid or solid filler, diluent, excipient, solvent or encapsulating material involved in carrying, formulating or transporting a chemical agent. Specific examples are water, glycols, oils, alcohols and the like in the case of oral liquid preparations. In oral solid forms solid carriers such as starches, sugars, kaolin, lubricants, binders, disintegrating agents and the like may be employed. Oral solid preparations are preferred over the oral liquid preparations. A preferred oral solid preparation is capsules and tablets, because of their ease of administration.

For parental compositions, the carrier will usually comprise sterile water, at least in large part, though other ingredients, to aid solubility for example, may be included. Injectable solutions, for example, may be prepared in which the carrier comprises PEG, saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wetting agent, optionally combined with suitable additives of any nature in minor proportions, which additives do not cause a significant deleterious effect on the skin. It is especially advantageous to formulate the aforementioned pharmaceutical compositions in dosage unit form for ease of administration and uniformity of dosage. Dosage unit form as used herein refers to physically discrete units suitable as unitary dosages, each unit containing a predetermined quantity of active ingredient(s) calculated to produce the desired therapeutic effect in association with the required pharmaceutical carrier.

Other embodiments of the present invention provide:

A pharmaceutical combination of a 5-HT4 receptor agonist and a cholinesterase inhibitor, or the pharmaceutically acceptable salts, racemates or enantiomers thereof, or a pharmaceutical composition comprising such a combination in the presence of a pharmaceutically acceptable carrier, as defined above, for use in the treatment of altered gastrointestinal motility, sensitivity and/or secretion and/or abdominal disorders.

Use of a pharmaceutical combination of a 5-HT4 receptor agonist and a cholinesterase inhibitor, or the pharmaceutically acceptable salts, racemates or enantiomers thereof, as defined above, in the manufacture of a medicament for use in the treatment of altered gastrointestinal motility, sensitivity and/or secretion and/or abdominal disorders.

Use of a 5-HT4 receptor agonist and a cholinesterase inhibitor, or the pharmaceutically acceptable salts, racemates or enantiomers thereof, as defined above, in the manufacture of a pharmaceutical composition for use in the treatment of altered gastrointestinal motility, sensitivity and/or secretion and/or abdominal disorders.

A method of treating a patient suffering from altered gastrointestinal motility, sensitivity and/or secretion and/or abdominal disorders comprising administering a therapeutically effective amount of a pharmaceutical combination of a 5-HT4 receptor agonist and a cholinesterase inhibitor, or the pharmaceutically acceptable salts, racemates or enantiomers thereof, or of a pharmaceutical composition comprising such a combination in the presence of a pharmaceutically acceptable carrier, e.g. as defined above, to the patient.

Generally, the pharmaceutical combinations or compositions of the present invention are employed for the treatment of altered gastrointestinal motility, sensitivity and/or secretion and/or abdominal disorders including, but not limited to, heartburn, bloating, postoperative ileus, abdominal pain and discomfort, early satiety, epigastric pain, nausea, vomiting, burbulence, regurgitation, intestinal pseudoobstruction, anal incontinence, GERD, IBS, dyspepsia, chronic constipation or diarrhea, gastroparesis, e.g. diabetic gastroparesis, ulcerative colitis, Crohn's disease, ulcers or the visceral pain associated therewith. In addition, the pharmaceutical combinations and compositions of the invention may also be employed as laxatives, as a preparation for a patient for colonoscopy, or as a means of regulating, stabilizing or normalizing gastrointestinal disorders, through for example, regulation, stabilization or normalization of enterochromaffin cell functions, GI secretion, afferent and efferent fiber activity or abdominal smooth muscle cell activity. The pharmaceutical combinations and compositions of the invention may also be useful in the treatment of menstrual cramps or spastic or interstitial cystitis.

The therapeutically effective dosage of the pharmaceutical compositions of this invention will vary with the severity of the condition to be treated, and the route of administration. The dose, and perhaps the dose frequency, will also vary according to the age, body weight, and response of the individual patient. In general, the combination of the 5-HT₄ receptor agonist and the cholinesterase inhibitor may be administered in a molar ratio having a range of from about 0.01 to about 2 for the 5-HT₄ receptor agonist to a range of from about 0.01 to 1000 for the cholinesterase inhibitor. As an example, the molar ratio for the 5-HT₄ receptor agonist to the cholinesterase inhibitor is about 1:1000 (first agent to cholinesterase inhibitor). As a more specific example, the molar ratio for the 5-HT₄ receptor agonist to the cholinesterase inhibitor may be about 1:1000, 1:500, 1:200, 1:100, 1:20, 1:5, 1:1 or 1:0.01. A preferable molar ratio is about 1:20, even more preferably about 1:5 and most preferably about 1:1.

The total daily dose range, which comprises the above-described molar ratio, for the conditions described herein, may be administered in a range of from about 0.01 mg to about 1000 mg. The daily dose range may be about 800 mg, 600 mg, 400 mg, 200 mg, 100 mg, 50 mg, 20 mg, 10 mg, 5 mg, 1 mg, 0.1 mg or 0.01 mg. Preferably, a daily dose range should be between about 0.5 mg to about 100 mg, while most preferably, a daily dose range should be between about 5 mg to about 75 mg. It is preferred that the doses are administered OD (once daily) or BID (2 times a day). In managing the patient, the therapy should be initiated at a lower dose, perhaps about 5 mg to about 10 mg, and increased up to about 50 mg or higher depending on the patient's response. It may be necessary to use dosages outside these ranges in some cases as will be apparent to those skilled in the art. Further, it is noted that the clinician or treating physician will know how and when to interrupt, adjust, or terminate therapy in conjunction with individual patient response. The term “therapeutically effective amount” is encompassed by the above-described molar ratio and dosage amounts and dose frequency schedule. A “therapeutically effective amount” can be administered in both a fixed or non-fixed combination of a 5-HT₄ receptor agonist, e.g. tegaserod, and a cholinesterase inhibitor.

### EXAMPLES

In vitro and in vivo testing of the effect of the combination of a 5-HT₄ receptor agonist and an acetylcholinesterase inhibitor upon gastrointestinal and colonic motility is performed using a range of methods to quantify the rate of transit of material through the GI tract. Gastric emptying time and gastrointestinal transit time (Mawe et al, 1989, Am J Physiol 282:G948; Jung et al, 2000, J Pharm Pharmacol 52:1031-1036; Trudel et al, 2002, Am J Physiol 282:G948) and colonic motility (Osinki et al, 1999, Am J Physiol, 276:G125-G131) is assessed in rodents.

Combinations according to the present invention demonstrate a positive effect upon gastrointestinal and colonic motility according to the above.

Background: Cholinergic stimulation is a common mechanism of action of most gastrointestinal (GI) prokinetics. Current therapeutic approaches to treat slow GI transit include the use of cholinesterase inhibitors that prevent the breakdown of acetylcholine (ACh) following its release from cholinergic nerve terminals. These agents augment the motor activity of the bowel, especially the colon, and are often used to stimulate colonic transit in patients with pseudo-obstruction, post-operative ileus and colonic inertia. Another approach used to treat chronic constipation is to use 5HT₄ receptor agonists, such as tegaserod, to stimulate GI transit via a mechanism involving presynaptic facilitation of ACh release. Since these two agents work independently on either side of the synapse to elevate synaptic levels of ACh, we aimed to investigate the potential synergy that may occur with a combination of the two agents in the amplification of cholinergic neurotransmission and thus GI transit.

Methods: In male rats (200-300 g) fecal pellet output was measured every 30 mins for 2 hr following administration of tegaserod (0.01-1.0 mg/kg i.p.), neostigmine (0.01-0.1 mg/kg i.p.) or a combination of both compounds.

Results: When dosed alone, tegaserod or neostigmine caused a dose and time-dependent increase in fecal pellet output. In combination, the lower doses of the two agents, which did not produce significant effects alone, increased fecal pellet output that was significantly different from the sum of the two compounds alone (Table 1). This synergistic effect was not seen at the higher doses of each compound, which may be explained by a bell shaped dose response in this model.

### SUMMARY & CONCLUSION

When combined, tegaserod and neostigmine synergize in the production of their stimulatory effect on colonic transit. This synergistic effect of a combination of a 5HT₄ receptor agonist and an anti-cholinesterase agent may prove to be a useful therapeutic approach treating conditions associated with slow colonic transit.

TABLE 1
Effect of Low Dose Tegaserod (Teg) or Neostigmine (Neo)
alone or in Combination on Fecal Pellet Output.

| Treatment | | # Fecal Pellets | | | |
|---|---|---|---|---|---|
| mg/kg i.p. | n | 30 min | 60 min | 90 min | 120 min |
| Teg 0.01 | 6 | 0.17 ± 0.17 | 0.17 ± 0.17 | 0.33 ± 0.21 | 1.50 ± 0.62 |
| Neo 0.01 | 3 | 0.67 ± 0.33 | 1.00 ± 0.58 | 1.00 ± 0.58 | 1.33 ± 0.67 |
| Teg & Neo | 12 | 1.83 ± 0.42* | 2.25 ± 0.41** | 2.67 ± 0.38*** | 3.58 ± 0.40*** |
| Teg 0.1 | 12 | 0.83 ± 0.32 | 1.25 ± 0.35 | 1.50 ± 0.34 | 1.83 ± 0.39 |
| Neo 0.01 | 3 | 0.67 ± 0.33 | 1.00 ± 0.58 | 1.00 ± 0.58 | 1.33 ± 0.67 |
| Teg &Neo | 12 | 2.25 ± 0.55 | 2.83 ± 0.58* | 3.33 ± 0.61* | 4.00 ± 0.70** |

Sum of tegaserod and neostigmine alone vs. teagerod plus neostigmine
*P < 0.05 and
**P < 0.01
***P < 0.001 using 2-way ANOVA and Bonferroni post tests.

1. A pharmaceutical combination, comprising:
a) a 5-HT4 receptor agonist, or a pharmaceutically acceptable salt, racemate or enantiomer thereof; and
b) a cholinesterase inhibitor, or a pharmaceutically acceptable salt, racemate or enantiomer thereof.

2. A pharmaceutical combination according to claim 1 which is synergistic.

3. A pharmaceutical combination according to claim 1 comprising
a) a 5-HT4 receptor agonist which is a compound of formula I 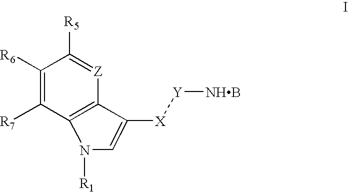
wherein
R₁ is hydrogen; C₁₋₆alkyl; (C₁₋₆alkyl)carbonyl; benzoyl; or phenylC₁₋₄alkyl-carbonyl;
R₅ is hydrogen; halogen; C₁₋₆alkyl; hydroxy; nitro; amino; C₁₋₆alkylamino; C₁₋₁₀alkyl-carbonylamino; C₂₋₆alkoxycarbonyl; SO₂NRₐR_{b} wherein each of Rₐ and R_{b} independently is hydrogen or C₁₋₆alkyl; cyano; or trimethylsilyl; C₁₋₆alkyl substituted by —SO₂—C₁₋₆alkyl, —SO₂NRₐR_{b}, —CONRₐR_{b}, —NH—SO₂—C₁₋₆alkyl, —N(C₁₋₆alkyl)-SO₂—(C₁₋₆alkyl), —NRₐR′_{b} wherein R′_{b} is hydrogen or C₁₋₆alkyl, C₂₋₆alkoxycarbonyl or —PO(C₁₋₄alkyl)₂; carboxy; CONRₐR′_{b}; —PO(C₁₋₆alkyl)₂; OCONR_{c}R_{d}, wherein each of R_{c} and R_{d} independently is C₁₋₆alkyl;
R₆ is hydrogen or, when R₅ is OH, R₆ is hydrogen or halogen,
Z is —CR₄═ wherein R₄ is hydrogen, halogen, hydroxy or C₁₋₆alkyl or, when R₅ is hydrogen or hydroxy, Z is also —N═,
R₇ is hydrogen, halogen, C₁₋₆alkyl or C₁₋₆alkoxy,
X-Y is —CR₈═N— or —CH(R₈)—NH— wherein R₈ is hydrogen or C₁₋₆alkyl, and
B is a radical of formula (a) or (b), 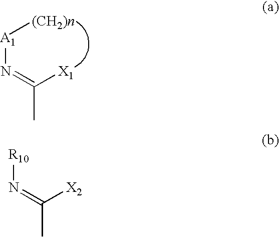
wherein
n is 1 or 2,
A₁ is C═O or CH₂,
X₁ is S; NR₁₁ wherein R₁₁ is hydrogen, (C₁₋₆alkyl)carbonyl, benzoyl or phenylC₁₋₄alkyl-carbonyl; or CR₁₂R₁₃ wherein each of R₁₂ and R₁₃ independently is hydrogen or C₁₋₄alkyl,
R₁₀ is hydrogen; C₁₋₁₂alkyl; C₁₋₆alkyl substituted by hydroxy, aryl, aryloxy, adamantyl, a heterocyclic radical, —NR₁₅—CO—R₁₆ or —NH—SO₂-aryl; C₅₋₇cycloalkyl; adamantyl; (C₁₋₁₀alkyl)carbonyl; benzoyl; phenyl(C₁₋₄alkyl)carbonyl; or —CONHR₁₄,
wherein
R₁₄ is C₁₋₁₀alkyl or C₅₋₇cycloalkyl,
R₁₅ is hydrogen or C₁₋₄alkyl, and
R₁₆ is C₁₋₆alkyl, C₅₋₇cycloalkyl, C₅₋₇cycloalkyl-C₁₋₄alkyl, aryl or arylC₁₋₄alkyl, wherever “aryl” appears as is or in the significances “aryloxy”, “—NH—SO₂-aryl” or “aryl(C₁₋₄alkyl)” in the above definition, it is phenyl or phenyl substituted by halogen, C₁₋₄alkyl or C₁₋₆alkoxy; and
wherever “heterocyclic radical” appears in the above definition, it is pyridyl, imidazolyl, benzimidazolyl, pyrrolidinyl, pyrrolidonyl, piperidino, pyrazinyl, perhydroindolyl or a radical of formula (c), (d) or (e) 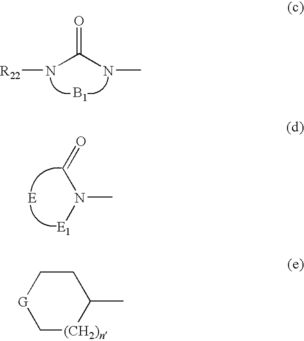
wherein
R₂₂ is hydrogen or C₁₋₄alkyl,
B₁ is —CH₂CH₂—, —COCH₂— or —(CH₂)₃— in which one or two H thereof can by replaced by C₁₋₄alkyl, or 1,2-phenylene,
E is —CH₂—CH₂—, —CH₂N(R₁₇)— or —(CH₂)₃— in which one or two H thereof can be replaced by C₁₋₆alkyl, or 1,2-phenylene,
E₁ is CO or CH₂,
R₁₇ is hydrogen or C₁₋₄alkyl,
G is CO, —CHCOOR₁₈, —CHCOR₁₉, 5,5-dimethyl-1,3-dioxan-2-ylidene or 1,3-dioxolan-2-ylidene, wherein R₁₈ is hydrogen or C₁₋₆alkyl and R₁₉ is C₁₋₆alkyl, and
n′ is 0 or 1, and
X₂ is —SR₂₀ or —NR₃R′₁₀ wherein R₂₀ is C₁₋₆alkyl, R₃ is hydrogen or C₁₋₆alkyl and R′₁₀ has one of the significances given for R₁₀ above, or R₃ and R′₁₀ together with the nitrogen atom to which they are attached form a heterocyclic radical as defined above;
with the proviso that where B is a radical of formula (b), only one of R₁₀ and R′₁₀ can be other than hydrogen and X₂ can be —SR₂₀ only when R₁₀ is hydrogen,
and a physiologically-hydrolysable and -acceptable ether or ester thereof when R₅ is hydroxy,
in free form or in pharmaceutically acceptable salt form, and
b) a cholinesterase inhibitor, or a pharmaceutically acceptable salt, racemate or enantiomer thereof.

4. A pharmaceutical combination according to claim 1 wherein the cholinesterase inhibitor is selected from Donepezil HCl, Rivastigmine Tartrate, Pyridostigmine Bromide and Galanthamine Hydrobromide.

5. A pharmaceutical combination according to claim 1 wherein the 5-HT₄ receptor agonist a) is tegaserod, in free form or in pharmaceutically acceptable salt form.

6. A pharmaceutical combination according to claim 1 for use in the treatment of altered gastrointestinal motility, sensitivity and/or secretion and/or abdominal disorders.

7. A pharmaceutical combination according to claim 1 where the dose range of the 5-HT₄ receptor agonist is about or exactly 0.01-0.1 mg/Kg.

8. A pharmaceutical combination according to claim 1 where the dose range of the cholinesterase inhibitor is about or exactly 0.01-0.1 mg/Kg.

9. A pharmaceutical combination according to claim 7 where the dose of the 5-HT₄ receptor agonist is about or exactly 0.01 mg/Kg.

10. A pharmaceutical combination according to claim 8 where the dose of the cholinesterase inhibitor is about or exactly 0.01 mg/Kg or 0.1 mg/Kg.

11. A pharmaceutical composition comprising the pharmaceutical combination according to claim 1 and a pharmaceutically acceptable carrier.

12. (canceled)

13. A method of treating a patient suffering from altered gastrointestinal motility, sensitivity and/or secretion and/or abdominal disorders comprising administering to the patient a therapeutically effective amount of a pharmaceutical combination according to claim 1 or a pharmaceutical composition according to claim 11.
